(19) United States
(12) Patent Application Publication
(10) Pub. No.: US 2023/0105998 A1
(43) Pub. Date: Apr. 6, 2023

(54) METHOD AND SYSTEM FOR GENERATING TASK-RELEVANT STRUCTURAL EMBEDDINGS FROM MOLECULAR GRAPHS

(71) Applicant: Huawei Cloud Computing Technologies Co., Ltd., Gui'an New District (CN)

(72) Inventors: Oleksandr YAKOVENKO, Vancouver (CA); Lei ZHANG, Shanghai (CN); Chi XU, Shanghai (CN); Nan QIAO, Beijing (CN); Yong ZHANG, Richmond (CA); Lanjun WANG, Shenzhen (CN)

(21) Appl. No.: 18/062,561

(22) Filed: Dec. 6, 2022

Related U.S. Application Data
(63) Continuation of application No. PCT/CN2021/091178, filed on Apr. 29, 2021.

(51) Int. Cl. G16C 20/20; G06N 3/08; G06N 3/0442; G16C 20/30; G16C 20/70

(52) U.S. Cl. CPC G16C 20/20; G06N 3/08; G06N 3/0442; G16C 20/30; G16C 20/70

(57) ABSTRACT

Methods and systems for generating embeddings from molecular graphs, which may be used for classification of candidate molecules. A physical model is used to generate a set of task-relevant feature vectors, representing local physical features of the molecular graph. A trained embedding generator is used to generate a set of task-relevant structural embeddings representing connectivity among the set of vertices and task-relevant features of the set of vertices. The task-relevant feature vectors are combined with the task-relevant structural embeddings and provided as input to a trained classifier. The trained classifier generates a predicted class label representing a classification of the candidate molecule.

### CROSS-REFERENCE TO RELATED APPLICATIONS

The present disclosure is a continuation application of International Application No. PCT/CN2021/091178 entitled “METHOD AND SYSTEM FOR GENERATING TASK-RELEVANT STRUCTURAL EMBEDDINGS FROM MOLECULAR GRAPHS”, filed Apr. 29, 2021, the entirety of which is hereby incorporated by reference.

### FIELD

Examples of the present disclosure relate to methods and system for generating embeddings from geometric graphs, including generating embeddings from molecular graphs to be used for computer-assisted prediction of molecular interactions, such as in computational molecular design applications.

### BACKGROUND

A molecular graph is a representation of the physical structure of a molecule. Atoms are represented as vertices of the molecular graph, and chemical bonds are represented as edges of the molecular graph. A molecule (and hence the representative molecular graph) can exhibit local symmetry, meaning that there are two or more sub-structures in the molecular that are substantially identical to each other on a local basis (e.g., on the basis of immediately local bonds). Unlike some other types of geometrical graphs (e.g., social graphs), molecular graphs can have many non-unique vertices with non-unique local connections.

In the field of drug design and in other biomedical applications, molecular symmetry can be important. For example, amino acids can have L and D enantiomers, which are non-superposable mirror images of each other, and which can have different activity levels. However, accounting for local symmetry in molecular graphs remains a challenge in developing machine learning-based techniques for drug design.

Accordingly, it would be useful to provide a solution to enable accurate representations of geometrical graphs (including molecular graphs) that have local symmetry, which can be used as input to machine learning-based systems.

### SUMMARY

In various examples, the present disclosure describes methods and systems for generating a set of embeddings to represent a molecular graph having local symmetry. A molecular graph representing a candidate molecule may be received into an embedding generator. The embedding generator outputs a set of embeddings that provides information about the structure of the molecular graph together with information about task-relevant features of the graph vertices. In parallel with generation of the set of embeddings, a physical model also generates a set of feature vectors representing the physical features of the graph vertices. Each embedding may be concatenated with a respective feature vector and provided as input data to a classifier, which predicts a class label for the candidate molecule.

The disclosed methods and systems may enable information about the structure of chemical compounds to be encoded with higher accuracy and precision than some existing techniques. The disclosed methods and systems may enable a trained classifier to generate more accurate predictions of class labels for candidate molecules (e.g., to classify molecules as active or inactive), which may be useful for molecular design applications (e.g., for drug design).

Although the present disclosure describes examples in the context of molecular graphs and molecular design applications, examples of the present disclosure may be applied in other fields. For example, any application in which data can be represented as geometric graphs, such as applications relating to social networks, city planning, or software design, may benefit from examples of the present disclosure.

The disclosed methods and systems may be applied as part of a larger machine learning-based system, or as a stand-alone system. For example, the disclosed system for generating embeddings may be trained by itself and the trained system used to generate embeddings, as data for training or input to a separate machine learning-based system (e.g., a system designed to learn and apply a chemical language model). The disclosed system for generating embeddings may also be integrated in a larger overall machine learning-based system and trained together with the larger system.

In some example aspects, the present disclosure describes a method for classifying a candidate molecule. The method includes: obtaining input data representing a molecular graph defined by a set of vertices and a set of edges, the molecular graph being a representation of the candidate molecule; generating, using a physical model, a set of feature vectors from the input data, the set of feature vectors representing local physical features of the molecular graph; generating, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule; combining each feature vector in the set of feature vectors with a respective task-relevant structural embedding in the set of task-relevant structural embeddings, to obtain a set of combined vectors; and generating, using a trained classifier, a predicted class label for the input data from the set of combined vectors, the predicted class label representing a classification of the candidate molecule.

In any of the preceding examples, the embedding generator may include a geometrical embedder based on good edit similarity and a gated recurrent unit (GRU) module, the geometrical embedder generating a set of geometrical embeddings representing the connectivity among the set of vertices, the GRU module further generating the set of task-relevant structural embeddings from the set of geometrical embeddings and task-relevant (e.g., physical) features.

In any of the preceding examples, the geometrical embedder may be trained to generate the set of geometrical embeddings using a hierarchy of margins to encode local connections with respect to each vertex in the set of vertices.

In any of the preceding examples, training the geometrical embedder and the GRU module may include generating, using a decoder neural network, a reconstructed adjacency matrix of the molecular graph from the set of task-relevant structural embeddings, computing a molecular structure reconstruction loss between the reconstructed adjacency matrix and an actual adjacency matrix of the molecular graph, and backpropagating the molecular structure reconstruction loss to update weights of the GRU module and geometrical embedder.

In any of the preceding examples, the molecular structure reconstruction loss may be used as a regularization term for training of the classifier.

In any of the preceding examples, combining each feature vector in the set of feature vectors with the respective task-relevant structural embedding in the set of task-relevant structural embeddings may include concatenating each feature vector in the set of feature vectors with the respective task-relevant structural embedding in the set of task-relevant structural embeddings.

In any of the preceding examples, the physical model may be a molecular docking model.

In some example aspects, the present disclosure describes a device for classifying a candidate molecule. The device includes a processing unit configured to execute instructions to cause the device to: obtain input data representing a molecular graph defined by a set of vertices and a set of edges, the molecular graph being a representation of the candidate molecule; generate, using a physical model, a set of task-relevant feature vectors from the input data, the set of task-relevant feature vectors representing local physical features of the molecular graph; generate, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule; combine each task-relevant feature vector in the set of task-relevant feature vectors with a respective task-relevant structural embedding in the set of task-relevant structural embeddings, to obtain a set of combined vectors; and generate, using a trained classifier, a predicted class label for the input data from the set of combined vectors, the predicted class label representing a classification of the candidate molecule.

In any of the preceding examples, the processing unit may be configured to execute instructions to cause the device to perform any of the preceding methods.

In any of the preceding examples, the physical model, the trained embedding generator and the trained classifier may be part of a molecule classification module executed by the processing unit.

In some example aspects, the present disclosure describes a computer-readable medium having instructions encoded thereon. The instructions, when executed by a processing unit of a device, cause the device to: obtain input data representing a molecular graph defined by a set of vertices and a set of edges, the molecular graph being a representation of the candidate molecule; generate, using a physical model, a set of task-relevant feature vectors from the input data, the set of task-relevant feature vectors representing local physical features of the molecular graph; generate, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule; combine each task-relevant feature vector in the set of task-relevant feature vectors with a respective task-relevant structural embedding in the set of task-relevant structural embeddings, to obtain a set of combined vectors; and generate, using a trained classifier, a predicted class label for the input data from the set of combined vectors, the predicted class label representing a classification of the candidate molecule.

In some example aspects, the present disclosure describes a computer-readable medium having instructions encoded thereon, wherein the instructions, when executed by a processing unit of a device, cause the device to perform any of the example methods described above.

### BRIEF DESCRIPTION OF THE DRAWINGS

Reference will now be made, by way of example, to the accompanying drawings which show example embodiments of the present application, and in which:

FIG. 1 illustrates an example molecule exhibiting local symmetry;

FIG. 2 is a block diagram illustrating an example molecule classification module including an embedding generator, in accordance with some embodiments of the present disclosure;

FIG. 3 illustrates some implementation details of an example embedding generator, in accordance with some embodiments of the present disclosure;

FIG. 4 illustrates an example or margins hierarchy in the context of a molecule, in accordance with some embodiments of the present disclosure;

FIG. 5 is a flowchart of an example method for training an embedding generator, in accordance with some embodiments of the present disclosure; and

FIG. 6 is a flowchart of an example method for classifying a molecular graph using the molecule classification module of FIG. 2, in accordance with some embodiments of the present disclosure.

Similar reference numerals may have been used in different figures to denote similar components.

### DESCRIPTION OF EXAMPLE EMBODIMENTS

The following describes technical solutions of this disclosure with reference to accompanying drawings.

The methods and systems described in examples herein may be used for generating embeddings to represent geometric graphs, in particular non-linear graphs having local symmetry, such as molecular graphs representing candidate molecules.

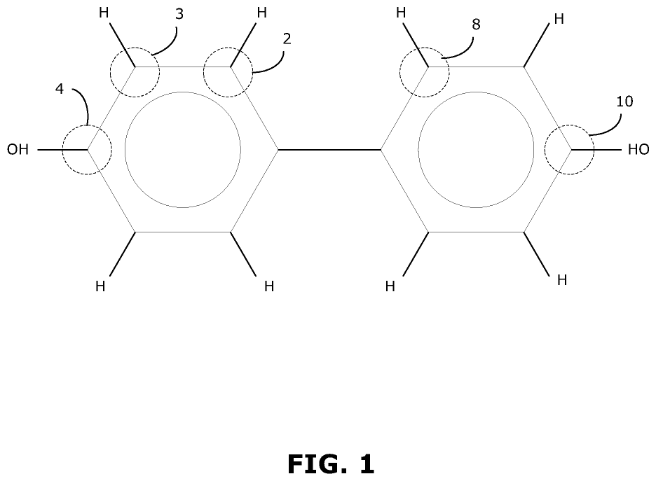

FIG. 1 illustrates an example small organic molecule (in this example, biphenyl) that exhibits local symmetry, for example at locations 2, 3, 4, 8 and 10 as indicated. Because of the local symmetry, it is difficult to design a machine learning-based technique that can consistently and accurately predict the answer to structural questions such as: whether the carbon at location 3 and the carbon at location 2 are connected; or whether the carbon at location 3 and the carbon at location 8 are connected (note that locations 2 and 8 are identical on a local level). Similarly, the chemical bonds of the carbon at location 4 are identical to the chemical bonds of the carbon at location 10 at a local level, but the carbons are not the same atoms. Such small organic molecules are of interest in many drug design applications. The disclosed methods and systems provide the technical effect that the structure of a geometric graph having local symmetry can be represented with little or no ambiguity, using a machine learning-based technique.

In the context of molecular modeling and drug design, the disclosed methods and systems enable more accurate and precise representation of the structure of a molecule, to enable a machine learning-based system to more accurately predict a class label for the molecule.

To assist in understanding the present disclosure, a general overview of conventional computational drug design techniques is first provided below.

In an existing drug design technique (e.g., as described by Wallach et al., “AtomNet: A Deep Convolutional Neural Network for Bioactivity Prediction in Structure-based Drug Discovery” arXiv:1510.02855v1), the screening of potential drug candidates begins with input of a dataset of molecular structures (e.g., in structure data file (SDF) format) of candidate molecules. The input dataset is processed using a physical model to generate feature data (e.g., in the form of feature vectors) for the respective molecular structures. The physical model is a machine learning-based model that simulates real-world characteristics of a molecule. For example, the physical model may be in the form of molecular docking, which models how a candidate molecule structurally binds (or “docks”) with a protein, based on the respective three-dimensional (3D) structures. Because molecular docking is concerned with how local features of a candidate molecule interacts with local features of a protein, the feature data generated based on molecular docking may represent local structures of a candidate molecule. The generated feature data are then used as input in a trained classifier, which performs a classification task to predict a class label for the candidate molecule. For example, the classifier may be trained to perform binary classification to classify the candidate molecule into potentially active or inactive class. The candidate molecules that have been classified as potentially active may then be subjected to further research and study. However, it should be noted that in this existing approach, higher-level (e.g., global) structural information of the candidate molecules (e.g., representations of the corresponding molecular graphs) are not provided as inputs to the classifier.

Another existing technique for drug design (e.g., as described by Zhavoronkov et al., “Deep learning enables rapid identification of potent DDR1 kinase inhibitors”, Nature Biotechnology, DOI:10.1038/s41587-019-0224-x) uses reinforced learning feedback to help improve the generation of candidate molecules by a learned molecular structure generator or selector. However, this approach also does not provide higher-level structural information to the classifier neural network.

An existing approach for generating symmetry-aware embeddings from a molecular graph (e.g., as described by Lee et al., “Learning compact graph representations via an encoder-decoder network”, Appl Netw Sci 4, 50 (2019) doi:10.1007/s41109-019-0157-9) uses a random walk approach. Random walk is a technique for generating a plurality of linear sequences from a non-linear graph, by starting at a random vertex of the graph and randomly selecting edges to follow, until a predefined sequence length has been generated (i.e., a predefined number of vertices has been traversed). The resulting linear sequences represent probabilistic graph connectivity. However, the probabilistic nature of random walks means that the overall structure of the non-linear graph is not represented uniformly (i.e., some vertices having a high number of connections may be overrepresented and other vertices having a low number of connections may be underrepresented), and there is a possibility that some vertices are not represented at all in the random walks (e.g., in the case of a very large molecule, some vertices may be unreachable within the predefined sequence length; or some vertices may not be reached by a random walk simply due to probability). Accordingly, the random walk approach may not be a reliable technique for generating embeddings from a molecular graph.

In the present disclosure, example methods and systems are described that generate a set of embeddings that represents information about higher-level features of a molecule to be provided as input to a classifier, together with feature data (e.g., generated by a physical model) representing more local physical features of the molecule. Because the input to the classier includes higher-level (i.e., less localized) structural information in addition to lower-level (i.e., more localized) feature data, the classifier may be able to generate predictions with higher accuracy, compared to some existing techniques.

The present disclosure provides methods and systems that use a machine learning-based embedding generator to generate a set of embeddings from a molecular graph. The embedding generator encodes the molecular graph into a set of embeddings that represents the high-level structure of the molecular graph together with task-related features of the graph vertices. Each embedding in the set of embeddings may be combined (e.g., concatenated) with a corresponding task-relevant feature vector in a set of task-relevant feature vectors, generated by a physical model, that represents more localized physical features of the graph vertices (e.g., a physical model representing task-relevant molecular interactions). The combined vectors may be used as input to a classifier, to predict a class label for the molecular graph.

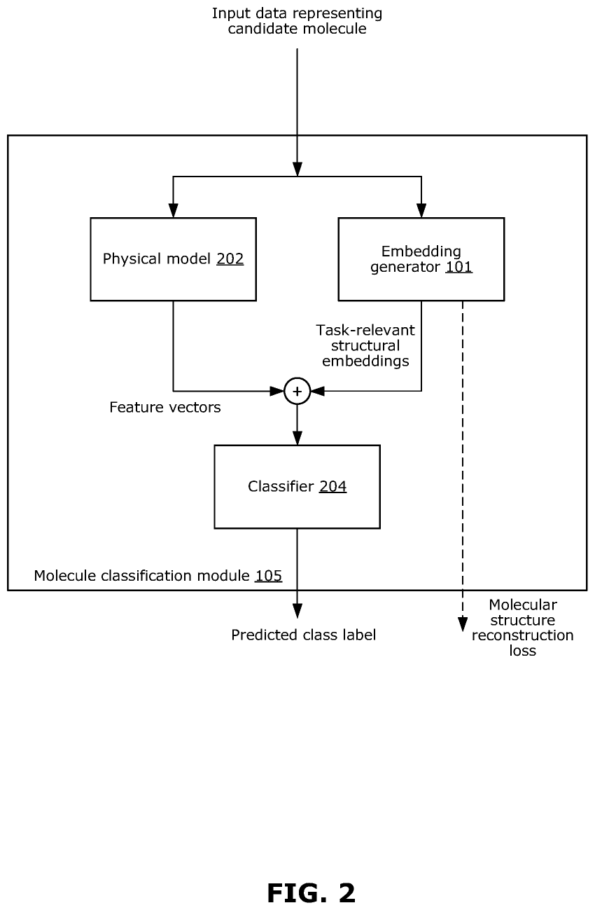

FIG. 2 is a block diagram illustrating an example of a disclosed embedding generator 101 applied in the context of a molecular classification module 105.

The molecular classification module 105 may be a software module (e.g., a set of instructions for executing a software algorithm), executable by a computing system. For example, a computing system may be a desktop computer, a workstation, a laptop, etc. The software module may be stored in a memory (e.g., a non-transitory memory, such as a read-only memory (ROM)) of the computing system. The computing system includes a processing unit (e.g., a neural processing unit (NPU), a tensor processing unit (TPU), a graphics processing unit (GPU) and/or a central processing unit (CPU)) that executes the instructions of the molecular classification module 105, to perform classification of a candidate molecule, as discussed below.

As shown in FIG. 2, the input to the molecular classification module 105 is input data representing a candidate molecule. For example, the input data may be a SDF representing a molecular graph of the candidate molecule. In the molecular graph, each vertex represents a corresponding atom of the candidate molecule, and each edge represents a corresponding chemical bond in the candidate molecule.

The input data (e.g., the SDF) is received by a physical model 202. The physical model 202 is a machine learning algorithm is designed to simulate (or model) the real-world characteristics of the candidate molecule. For example, the physical model 202 may be designed based on a model of molecular docking. The physical model 202 processes the input data to output a set of task-relevant feature vectors, where the set of task-relevant feature vectors is a latent representation of the local physical interactions that are computed by the molecular docking model. Each task-relevant feature vector is a latent representation of the physical features local to a respective atom of the candidate molecule (where each atom is a vertex of the molecular graph), where the physical features are relevant to real-world molecular interactions.

The input data is also received by the embedding generator 101. The embedding generator 101 processes the input data to output a set of embeddings, which is a latent representation of the structural connectivity of the molecular graph. The set of embeddings also represents task-relevant feature information, as discussed further below. Each embedding in the set of embeddings represents structure connectivity and task-relevant features of a respective vertex of the molecular graph.

The embedding for each vertex is combined (e.g., concatenated) with the respective task-relevant feature vector for that vertex to obtain a respective combined vector. The set of combined vectors is provided to a trained classifier 204. The classifier 204 outputs a predicted class label for the molecular graph (and thus classifying the candidate molecule). The classifier 204 may be a binary classifier, for example, that classifies the candidate molecule as being potentially active (and hence should be subjected to further study) or inactive (and hence does not require further study). It should be understood that the classifier 204 may be designed and trained to perform different classification tasks, depending on the application.

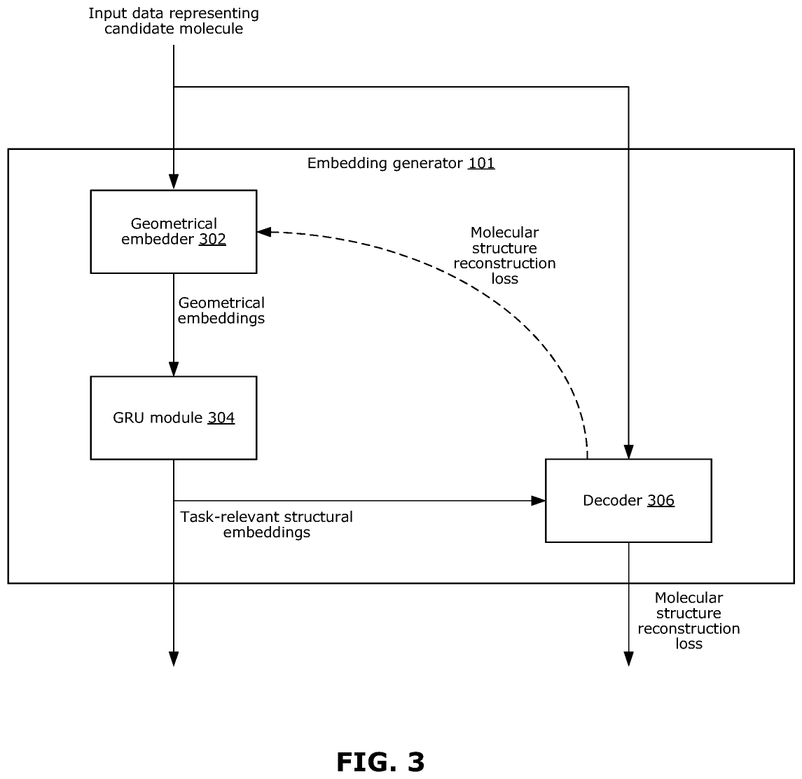

FIG. 3 illustrates details of the embedding generator 101, which may be part of the molecule classification module 105. In some examples, the embedding generator 101 may also be used as a standalone module, or as part of other modules aside from the molecule classification module 105.

To assist in understanding the present disclosure, some notations are introduced. A molecule may be represented in the form of a molecular graph, denoted as G(V_{graph},E_{graph}), where V_{graph} denotes a set containing all the vertices in the graph G and E_{graph} denotes a set containing all the edges connecting the vertices. For a molecular graph, the set of vertices V_{graph} represents chemical atoms (e.g., carbon, oxygen, etc.) and the set of edges E_{graph} represents chemical bond orders between atoms. In other non-molecular or non-biomedical contexts, the set of vertices V_{graph} and the set of edges E_{graph} may represent other features.

In the disclosed methods and systems, a function, denoted F, is modeled by the embedding generator 101 to generate a set of embeddings, denoted as vₑ. The set of embeddings vₑ (also referred to as structural embeddings) may be defined as vₑ={F(v,E_{graph})|v∈V_{graph}}. Each embedding in the set of embeddings vₑ is a k-dimensional vector, and each embedding corresponds to a respective vertex in the set of vertices V_{graph}. Thus, the set of embeddings vₑ forms an n-by-k matrix, where n is the number of vertices in the set of vertices V_{graph}, and k is the number of features per vertex. Each embedding represents structural features (e.g., connectivity). In particular, the set of embeddings vₑ may be a representation that can be decoded to reconstruct the first power of the graph adjacency matrix of the molecular graph, denoted as A(G) or simply A. As will be discussed further below, in other examples higher powers of the graph adjacency matrix A may be reconstructed using the set of embeddings vₑ.

The graph adjacency matrix A is a square matrix of size n×n, where n is the number of vertices in the set V_{graph}. An entry in the graph adjacency matrix A, denoted as aᵢⱼ, is 1 if there is an edge from the i-th vertex to the j-th vertex, and 0 otherwise. It should be noted that the graph adjacency matrix A is able to represent directional edges. For example, if aᵢⱼ is 1 and aⱼᵢ is 0, this would indicate that there is a unidirectional edge from the i-th vertex to the j-th vertex (i.e., there is no edge in the direction from the j-th vertex to the i-th vertex). A molecular graph may not have any unidirectional edges, however other types of geometric graphs (e.g., social graphs) may have unidirectional edges. The first power of the graph adjacency matrix A represents the direct connections between vertices, where a direct connection from the i-th vertex to the j-th vertex means that no other vertex is traversed.

The embedding generator 101 includes a geometrical embedder 302 and a gated recurrent unit (GRU) module 304. The embedding generator 101 is trained for each candidate molecule (e.g., each candidate molecule being classified by the molecule classification module 105). During training of the embedding generator 101, the embedding generator 101 also implements a decoder 306. The decoder 306 may be discarded or disabled during the inference phase of the trained embedding generator 101.

Input data representing the candidate molecule is received from a molecular structures storage (e.g., a SDF) and is projected into a latent space using the geometrical embedder 302. As will be discussed further below, the geometrical embedder 302 projects the input data into a binary classification latent space that classifies two samples as being local or not local to each other, based on good edit similarity. The geometrical embedder 302, which may also be referred to as a good edit similarity learning module, uses an approach based on good edit similarity, discussed further below, to generate a set of geometrical embeddings, that encodes (or more generally represents) connectivity between the vertices of the molecular graph. The geometrical embedder 302 generates the set of geometrical embeddings based on a hierarchy of geometrical margins in the latent space. Using a hierarchy of margins approach, the connectivity of a given vertex to each other vertex is classified as being local or not local to the given vertex, with each geometrical embedding representing the structural features local to each vertex. The result is a set of geometrical embeddings, where each geometrical embedding is a vector that encodes (or more generally represents) the structural features local to a respective vertex of the molecular graph in the form of Euclidian distances (i.e., margins) in the latent space.

The set of geometrical embeddings generated by the geometrical embedder 302 is processed by the GRU module 304. The GRU module 304 merges each geometrical embedding with task-relevant feature information, to output the set of task-relevant structural embeddings vₑ. For example, the bond order of each edge (e.g., single bond, double bond or triple bond) connected to a given vertex may be task-relevant feature information that is encoded into the task-relevant structural embedding for that vertex. Another example of task-relevant (or problem specific) features relevant to drug design classification goals are potential physical interactions of the given vertex, such as partial electric charge at the corresponding atom, its Van-der-Waals radius, hydrogen bonding potential etc. Thus, the geometrical embedder 302 outputs a latent representation of the geometric topology (i.e., vertices and connecting edges) of a geometric graph, and the GRU module 304 further extends this into a more abstract latent representation that is also relevant to the overall task (e.g., molecular classification) to be performed using the set of task-relevant structural embeddings vₑ. The set of task-relevant structural embeddings vₑ are outputted, and used as input by a classifier (see FIG. 3).

In the training phase, the set of task-relevant structural embeddings vₑ outputted by the embedding generator 101 are also processed by a decoder 306 to reconstruct the graph adjacency matrix. The reconstructed graph adjacency matrix (denoted as A′) can be compared with the graph adjacency matrix A (e.g., computed directly from the input data) to compute a molecular structure reconstruction loss. The molecular structure reconstruction loss can be used as a loss term for training of the entire molecule classification module 105. For example, the molecular structure reconstruction loss may be included as a regularization term for calculating the loss function for the classifier 204. For example, in the training phase of the classifier 204, a classification loss term may be computed. The molecular structure reconstruction loss may then be aggregated (as a regularization term) with the classification loss term, to arrive at a loss function that may be generally expressed as:

Loss=classification loss+weight*reconstruction loss

where the weight applied to the reconstruction loss is a hyperparameter. If the molecular structure reconstruction loss is included as a regularization term for training the classifier 204, the aim of the training may be to achieve good classification of the candidate molecule and at the same time constraining the task-relevant structural embeddings to correctly encode the structural information about the molecule.

The molecular structure reconstruction loss can also be used for training of the geometrical embedder 302. FIG. 3 illustrates how the gradient of the molecular structure reconstruction loss can be used to update (indicated by dashed curved arrow) the weights of the geometrical embedder 302. The molecular structure reconstruction loss may be computed, for example, based on the binary cross-entropy (BCE) loss between the reconstructed adjacency matrix A′ and the adjacency matrix A that is directly computed from the input data. Training using the molecular structure reconstruction loss may help to ensure the set of geometrical embeddings generated by the geometrical embedder 302 are an accurate representation of the molecular structure.

Details of the geometrical embedder 302 are now provided. The geometrical embedder 302 performs binary classification, based on a hierarchy of margins, to generate a set of geometrical embeddings that includes one geometrical embedding for each vertex in the molecular graph. Given the ith vertex vᵢ, the corresponding embedding vector el contains a binary value (e.g., a value of “1” or “0”) at the jth position indicating whether the jth vertex vⱼ is classified as local to the ith vertex vᵢ.

The geometrical embedder 302 is designed to perform binary classification based on a good edit similarity function. A good edit similarity function is based on the concept of edit similarity (or edit distance). Edit similarity is a way to measure similarity between two samples (e.g., two strings) based on the number of operations (or “edits”) required to transform a first sample to the second sample. The smaller the number of operations, the better the edit similarity. Good edit similarity is a characteristic that two samples are close to each other, according to some defined goodness threshold. A good edit similarity function is defined by the parameters (ϵ, γ, τ). The good edit similarity function formalizes a classifier function which guarantees that, if optimized, (1−ϵ) proportion of samples are, on average, 2γ times closer to a random sample of the same class than to a random “reasonable” sample of the opposite class; where at least a τ fraction of all samples are “reasonable”.

A good edit similarity function is defined for support vector machine (SVM) classifiers by Bellet et al. (“Good edit similarity learning by loss minimization” Mach Learn 89, 5-35 (2012) doi:10.1007/s10994-012-5293-8) as follows. The loss function that estimates classifier accuracy can be written as follows for the case of SVM classifiers:

$$L = min \frac{1}{N} \sum V ( C , {x}_{i} , {x}_{j} ) ) + β {‖ C ‖}_{F}^{2} \qquad ( 1 )$$

where L is the loss function, V is a projector function to map the coordinates of samples xᵢ and xⱼ into a latent space with some desired margin, N is a predefined number of “reasonable” samples, C is a set of learnable parameters (e.g., weights), and β is a selected regularization constant. The projector function V is the function to be learned, and maps coordinates of the samples xᵢ and xⱼ into a binary classification latent space that classifies the samples xᵢ and xⱼ as local or not local. The binary classification latent space separates the two classes (i.e., local or not local) by a defined margin. The margin is defined based on a desired separation between classes (which may be defined based on application). To obtain the desired margin between classes, the projector function V is defined to be a function of minimal edit distance of the feature vectors of xᵢ and xⱼ. In order to introduce the learnable parameters C and enable training of V, a transformer function E is applied to the input samples xᵢ and xⱼ. The resulting formulation is as follows:

$$V ( C , {z}_{i} , {z}_{j} ) = \{ \begin{matrix}{[ {B}_{1} - E ( C , {x}_{i} , {x}_{j} ) ]}_{+} if {l}_{i} \neq {l}_{j} \\ {[ E ( C , {x}_{i} , {x}_{j} ) - {B}_{2} ]}_{+} if {l}_{i} = {l}_{j}\end{matrix} \qquad ( 2 )$$

where the operation [⋅]₊ means only positive values are taken (i.e., [y]₊=max(y, 0)), I are class labels, and B₂ and B₂ are margin geometry defining constants. Somewhat simplified intuition behind equation (2) is that the aim is to find a coordinates transforming function E that tends to place input samples not only at the proper side of the ‘locality’ classification decision boundary but also at the desired distance (B₁ or B₂) from the boundary. In some sense the concept of good edit similarity function benefits from a built-in regularizer of the locality classification problem which additionally enforces similar items to stay similar with respect to the classifier decision boundary. The latent space distance constants B₁ and B₂ are expressed via a desired class separation margin γ as follows:

$$\begin{matrix}{B}_{1} = - ln ( \frac{1 - γ}{2} ) & {B}_{2} = - ln ( \frac{1 + γ}{2} )\end{matrix} \qquad ( 3 )$$

$$s . t . \begin{matrix}{B}_{1} \geq - ln ( \frac{1}{2} ) & 0 \leq {B}_{2} \leq - ln ( \frac{1}{2} )\end{matrix} \qquad ( 4 )$$

It should be noted that the definition of (ϵ, γ, τ)-good edit similarity function discussed by Bellet et al. is not designed for training neural networks, and is only applicable to vectors or sequences, not geometric graphs.

In the present disclosure, the concept of good edit similarity is adapted to enable latent representation of the structure of a geometric graph (e.g., a molecular graph). In particular, the present disclosure adapts good edit similarity to be applicable to non-linear graphs, by introducing a hierarchical structure to graph margins.

Equation (3) above defines the desired geometry of margins as a constant separation that is fixed at 2γ wide. In the present disclosure, the margins have been redefined to enable a variable margin, which is used to represent graph connectivity information. Specifically, the margin γ is redefined such that vertices that are local to each other are localized and classed together, and are separated by a margin γ from other vertices that are considered to be non-local. In particular, the margin γ is defined as a function of the distance matrix D:

γ=ƒ(D) (5)

where the distance matrix D (also referred to as the minimal pairwise graph distance matrix) is a matrix where the entry dᵢⱼ has a non-negative integer value representing the shortest distance to travel from the i-th vertex to the j-th vertex in the graph, where the distance is calculated as the number of vertices traversed from the i-th vertex to the j-th vertex (inclusive of the j-th vertex and exclusive of the i-th vertex). If i=j, then dᵢⱼ is zero. If the i-th vertex and the j-th vertex are directly connected to each other (with no vertex in between), then dᵢⱼ has the value 1. If there is no path between the i-th vertex and the j-th vertex (e.g., due to unidirectional connections in the graph), then dᵢⱼ is infinite or is undefined. The distance matrix D may be computed from the input data to the geometrical embedder 302, for example.

In the context of molecular graphs, the function ƒ may represent the separation criteria which defines the offset between the desired vertex location relative to the locality decision boundary, and means that only vertices (representing atoms) that are directly bonded to each other (i.e. dᵢⱼ=1) are classed together. Additionally, it is desirable for the function ƒ to be numerically stable. Based on equations (3) and (4) above, the following constraints apply:

$$\begin{matrix}0 \leq \frac{f ( D )}{2} < \frac{1}{2} \to f ( D ) ϵ [ 0 , 1 ) , & D ϵ [ 0 , + ∞ )\end{matrix} \qquad ( 6 )$$

The meaning of reformulated constraints in (6) is that a range of possible graph distances, which is [1 . . . +∞), needs to be mapped into [0 . . . 1) range to be compatible with concept of good edit similarity functions. An example definition of the function ƒ that satisfies the constraints in equation (6) is γ=ƒ(D)=π⁻¹ tan⁻¹ (D). Other definitions of the function ƒ may be found through routine testing, for example. Substituting this definition for the margin γ into equation (3) above results in the following:

$${B}_{1} = - ln ( \frac{1}{2} - \frac{{tan}^{- 1} ( D )}{π} ) \qquad ( 7 )$$

$${B}_{2} = - ln ( \frac{1}{2} + \frac{{tan}^{- 1} ( D )}{π} )$$

Equation (7) provides a hierarchy of margins. Conceptually, defining the margins in this way means that each given vertex (e.g., atom) in the graph is at the center of a margins hierarchy, and all vertices that are directly connected to the given vertex are assigned to the same class as the given vertex. The result is that directly connected vertices (e.g., atoms directly bonded to each other) are mapped close to each other in the latent space. Any vertices that are not directly connected to the given vertex are separated from the given vertex by a margin, which is a function of their pairwise distance (i.e., shortest path) in the geometric graph, and are not classed together with the given vertex.

Substitution of equation (7) into equation (2) and then into equation (1) provides the following loss function:

$$L = min \frac{1}{N} \sum \{ \begin{matrix}{[ - ln ( \frac{1}{2} - \frac{{tan}^{- 1} ( D )}{π} ) - exp ( {( {x}_{i} - {x}_{j} )}^{T} . C . ( {x}_{j} - {x}_{i} ) ) ]}_{+} & if {l}_{i} \neq {l}_{j} \\ {[ exp ( {( {x}_{i} - {x}_{j} )}^{T} . C . ( {x}_{j} - {x}_{i} ) ) + ln ( \frac{1}{2} + \frac{{tan}^{- 1} ( D )}{π} ) ]}_{+} & if {l}_{i} = {l}_{j}\end{matrix} + β {‖ C ‖}_{F}^{2}$$

This loss function can be used to compute gradients for training the geometrical embedder 302 in a latent space, with respect to the locally learnable embeddings (or feature vectors) x and the globally trainable parameters matrix C (i.e., the gradients

$$\frac{\partial L}{\partial x} and \frac{\partial L}{\partial C}$$

) for a given distance matrix D. The embeddings xᵢ and xⱼ are encoded for respective vertices of the graph (i.e., representing features of respective atoms of the candidate molecule). The matrix C encodes the penalty cost for editing the vector xᵢ into xⱼ. The intuition behind this computation is that an optimal context x, in which given structural information D can be encoded efficiently, is dependent on the structure itself and thus should found locally (i.e. the weights of x are specific to one particular candidate molecule) while the meaning of the latent space axes (i.e. the matrix C) is unified over the entire chemistry field and thus it is learned globally (i.e., not specific to any one candidate molecule).

The geometrical embedder 302 may have any suitable neural network architecture (e.g., a fully-connected neural network). Training of the geometrical embedder 302 using this loss function may be performed using any optimization technique, including any appropriate numerical method, such as the AdaDelta method. It may be noted that because the latent space is a convex function of x and C (due to the properties of good edit similarity, and the fact that the latent space is based on good edit similarity), any reasonable initialization of x may be used. For example, a set of random but unique {0, 1}-elements k-dimensional vectors of real numbers may be used as initialization of x.

As noted above, distance matrix D, which contains pairwise shortest distances between vertices of the graph, is required for computing the loss function. Any suitable technique may be used to compute the distance matrix D from the input data representing the geometric graph. For example, a suitable algorithm for computing the distance matrix D is described by Seidel, “On the All-Pairs-Shortest-Path Problem in Unweighted Undirected Graphs” J. Comput. Syst. Sci. 51(3):400-403 (1995).

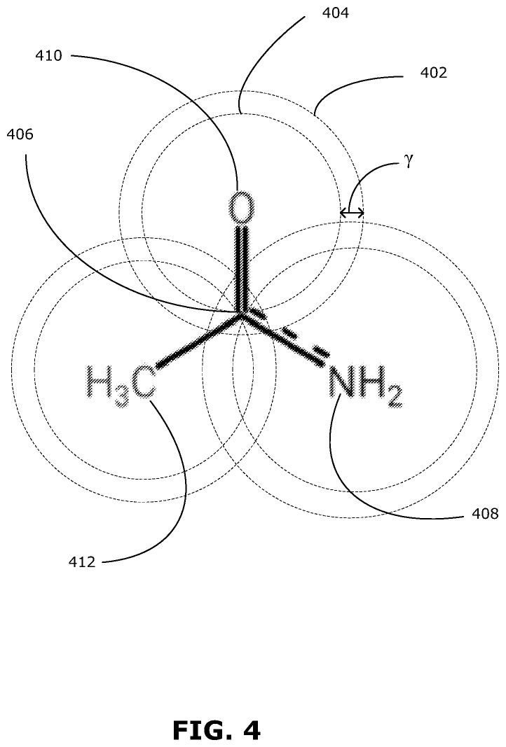

FIG. 4 illustrates an example of margins hierarchy, as defined above, in the case of an example small molecule, namely acetamide, in two-dimensional (2D) space.

In the case of acetamide (ignoring hydrogens for clarity), the distance matrix D may be represented as follows (note that the rows and columns have been labeled with each vertex, for ease of understanding):

| | N | CO | O | C4 |
|---|---|---|---|---|
| N | 0 | 1 | 2 | 2 |
| CO | 1 | 0 | 1 | 1 |
| O | 2 | 1 | 0 | 2 |
| C4 | 2 | 1 | 2 | 0 |

where N is the nitrogen at location 408, CO is the central carbon at location 406, O is the oxygen at location 410 and C4 is the carbon in the methyl group at location 412.

Then, the binary locality classification of the vertices relative to each other (i.e., if the label of vertex i, lᵢ, is the same as the label of vertex j, lᵢ, the value is “true”) may be represented as:

| | N | CO | O | C4 |
|---|---|---|---|---|
| N | True | True | False | False |
| CO | True | True | True | True |
| O | False | True | True | False |
| C4 | False | True | False | True |

Then the margin may be represented by the parameter γ as follows (where γ is half of the margin distance) and ArcTan of the matrix denotes matrix of element-wise arctangents:

$$γ = \frac{Arc Tan ( \begin{matrix}0 & 1 & 2 & 2 \\ 1 & 0 & 1 & 1 \\ 2 & 1 & 0 & 2 \\ 2 & 1 & 2 & 0\end{matrix} )}{π}$$

Consider the vertex representing the atom O (i.e., oxygen). The outer circle 402 defines the margin centered on the vertex O and the inner circle 404 indicates a distance that is γ apart from the margin and towards the vertex (it should be noted that the total margin width is two times γ; that is, the margin also extends a distance γ from the outer circle 402 away from the vertex). The inner circle 404 encompasses all atoms directly bonded to the vertex O (namely, the central carbon atom at location 406), and atoms that are not directly bonded to the vertex O are at least a distance of 2γ (i.e., the margin width) away from the inner circle 404. FIG. 4 similarly illustrates the margins for the vertex representing the atom N (i.e., nitrogen), and for the vertex representing the atom C (i.e., carbon). For compactness, the two hydrogen atoms (i.e., H₂) that are bonded to N are merged to the vertex N, and the three hydrogen atoms (i.e., H₃) that are bonded to C are merged to the vertex C. It may be noted that each vertex O, N and C are at a graph distance of two apart from each other, and each vertex O, N and C are at a graph distance of one from the central atom at location 406. This geometry is accurately represented by the use of margins. Specifically, the central atom at location 406 (which is directly connected to each vertex O, N and C) is within the distance γ from the margins of each vertex O, N and C; thus, the central atom is considered to be local to each of the vertex O, N and C. Each vertex O, N and C (each of which is not directly connect to any other of the vertices O, N and C) is farther than 2γ from the margins of the other vertices; thus, each vertex O, N and C is considered to be non-local to each of the other vertices. The use of hierarchy margins thus corresponds to Euclidian geometry optimization in a k-dimensional space with pairwise potentials between atoms (specifically, attractive potentials between two bonded atoms, or repulsive potentials between two non-bonded atoms) being represented by pairs-wise graph distances.

Reference is again made to FIG. 3. The geometrical embedder 302, as disclosed herein, enables the connectivity of all vertices to be uniformly represented in a set of geometrical embeddings (i.e., there are no overrepresented or underrepresented vertices in the geometrical embeddings). The loss function is defined based on a modified definition of good edit similarity (adapted to be applicable to geometric graphs). The loss function, as defined above, is a convex function, which may help to ensure that the weights of the geometrical embedder 302 will converge during training.

Details of the GRU module 304 are now discussed. The geometrical embedder 302 converts input data representing a non-linear geometric graph into a set of geometrical embeddings, representing the connectivity of the geometric graph (e.g., representing atoms in the candidate molecule) as a hierarchy of geometrical margins in latent space. The GRU module 304 receives the set of geometrical embeddings and further processes the set of geometrical embeddings to generate a set of latent representations, referred to as task-relevant structural embeddings, that encode task-specific feature information of each vertex as well as connectivity.

The GRU module 304 includes a GRU layer (denoted as GRU). It should be understood that other neural network architectures may be used, and any neural network that can be trained to learn a latent representation of task-relevant features may be used. For example, the GRU module 304 may be implemented using a long-short term memory (LSTM) neural network instead. In this example, the GRU module 304 additionally includes of two fully-connected layers denoted as H₀ and H. H₀ is used only at initialization, to translate the set of geometrical embeddings from the geometrical embedder 302 into the latent space of the GRU layer as follows:

hᵢ₀=H₀(vᵢ,eᵢ|θ_{H}_{₀})

where hᵢ₀ is the geometrical embedding for the i-th vertex translated into the latent space of the GRU layer, vᵢ is the vertex data, i.e. the concatenation of outputs from task-relevant physical model and of good edit similarity routine, eᵢ is the geometrical embedding for the i-th vertex, and θ_{H}_{₀} is the set of weights for H₀. The initial set of concatenated task-relevant geometrical embeddings is then propagated for a predefined number of iterations (e.g., N iterations, where N is some positive integer, which may be selected through routine testing) through the second layer H and the GRU layer GRU. In each iteration, the following computations are performed:

$${𝒳}_{i j} = \{ \begin{matrix}H ( {h}_{i} ∧ {h}_{j} ∧ {e}_{i} ∧ {e}_{j} \vert {θ}_{H} ) if {a}_{i j} \neq 0 \\ \vec{0} if {a}_{i j} = 0\end{matrix}$$

$${x}_{i} = {\sum }_{j} {𝒳}_{i j}$$

$${h}_{n + 1} = G R U ( x , {h}_{n} \vert {θ}_{G R U} )$$

where aᵢⱼ is an entry from the adjacency matrix indicating the adjacency of the i-th and j-th vertices, hᵢ and hⱼ are the learned task-relevant structural embeddings of the i-th and j-th vertices, respectively, eᵢ and eⱼ are the geometrical embeddings of the i-th and j-th vertices, respectively, θ_{H} is the set of weights for the layer H, θ_{GRU} is the set of weights for the GRU layer, χᵢⱼ is the output of layer H (an unrolled graph convolution operation) filtered using the adjacency matrix as a mask, and the symbol ∧ denotes a vector concatenation operation. Training at each iteration is performed jointly with the decoder 306, in which the backpropagation is based on the adjacency reconstruction loss from the decoder 306. At the end of N iterations, a set of final task-relevant structural embeddings, denoted as h_{N}, is obtained.

In the training phase, the set of task-relevant structural embeddings h_{N} is provided as input to the decoder 306, which performs pairwise concatenation of the embeddings (i.e., concatenates each pair of embeddings hᵢ and hⱼ, for all vertex pairs i≠j) and estimates the probability of a given pair of vertices to be connected. The decoder 306 may be implemented using a simple fully-connected network, denoted as G. The operation of the decoder 306 may be represented as follows:

gᵢⱼ=G(hᵢ∧hⱼ|θ)

where gᵢⱼ is the probabilistic adjacency value between the i-th and j-th vertices, and θ is the set of weights of G. The probabilistic adjacency values, computed for all pairs of vertices, together form the reconstructed adjacency matrix A′.

A loss (referred to as the molecular structure reconstruction loss) is computed between the reconstructed adjacency matrix A′ and the actual adjacency matrix A computed directly from the input data. In particular, the reconstructed adjacency value gᵢⱼ between the i-th and j-th vertices is compared to the corresponding adjacency value aᵢⱼ in the adjacency matrix A. The molecular structure reconstruction loss is computed using Binary Cross Entropy (BCE), as follows:

θ=argmin ΣᵢΣⱼBCE(gᵢⱼ,aᵢⱼ) where

BCE(x,y)=y·ln(x)+(1−y)·ln(1−x)

The computed loss is differentiable and may be used to update the parameters of the geometrical module 302 and the GRU module 304 using backpropagation, for example.

The trained GRU module 304 (e.g., after training has converged) outputs a set of task-relevant structural embeddings h_{N}, each task-relevant structural embedding hᵢ corresponding to a respective vertex vᵢ of the molecular graph. Each task-relevant structural embedding hᵢ encodes information about task-relevant features of the corresponding vertex vᵢ as well as the connectivity of the vertex vᵢ in the molecular graph.

The set of task-relevant structural embeddings h_{N} generated by the GRU module 304 may be provided as input to other neural networks. For example, as shown in FIG. 2, the task-relevant structural embeddings may be used as input, together with task-relevant feature vectors from the physical model 202, to the classifier 204.

In the example of FIG. 3, the decoder 306 is used to reconstruct the first power of the adjacency matrix A, to compute the molecular structure reconstruction loss during training. In other examples, higher powers of the adjacency matrix A may also be reconstructed, for example by using multiple decoders stacked on the same input. The molecular structure reconstruction loss may then be computed based on the higher powers of the adjacency matrix A, in addition to the first power of the adjacency matrix A. Training using molecular structure reconstruction loss computed from reconstructions of higher powers of the adjacency matrix A may help to improve the quality of the embeddings generated by the embedding generator 101.

The encoder-decoder architecture of the embedding generator 101 enables unsupervised training of the GRU module 304.

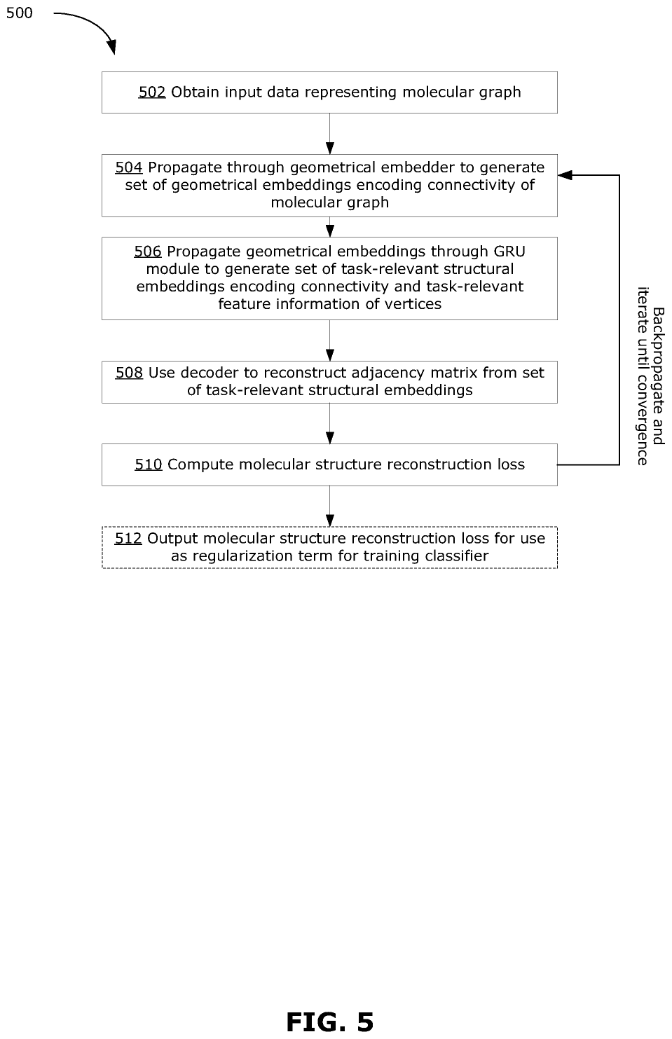

FIG. 5 is a flowchart illustrating an example training method 500 for training the embedding generator 101. The method 500 may be performed by any suitable computing system that is capable of performing computations for training a neural network.

At 502, input data representing a molecular graph (e.g., a candidate molecule) is obtained (e.g., an electronic file representing the molecular graph may be received as input data). For example, the input data may be a SDF representing the candidate molecule. The input data represents the set of vertices in the molecular graph and the set of edges connecting the vertices.

At 504, the input data is propagated through the geometrical embedder 302 to generate a set of geometrical embeddings encoding the connectivity among the vertices of the molecular graph. As described previously, the geometrical embedder 302 performs binary classification, based on good edit similarity and a hierarchy of margins, to encode the connectivity among the vertices of the molecular graph.

At 506, the set of geometrical embeddings is propagated through the GRU module 304 to generate a set of task-relevant structure embeddings encoding connectivity and task-relevant feature information of the vertices. As described previously, the GRU module 304 may include full-connected layers H₀ and H, as well as a GRU layer.

At 508, the set of task-relevant structural embeddings is propagated through the decoder 306 to reconstruct the adjacency matrix. For example, the decoder 306 may be implemented using a FCNN, which generates output representing the probabilistic adjacency between vertex pairs, as described above.

At 510, a loss function (e.g., a BCE loss) is computed using the reconstructed adjacency matrix and the ground-truth adjacency matrix of the molecular graph, to obtain a molecular structure reconstruction loss. The gradient of the molecular structure reconstruction loss is computed and backpropagated to update the weights of the geometrical embedder 302 and the GRU module 304 using gradient descent. Steps 506-510 may be iterated until a convergence condition is satisfied (e.g., a defined number of iterations have been performed, or the adjacency reconstruction loss converges). The trained weights of the geometrical embedder 302, GRU module 304 and optionally decoder 306 are stored.

Optionally, at 512, the molecular structure reconstruction loss may be outputted to be used as a regularization term in the loss function for training a classifier (e.g., the classifier 204 in FIG. 2). It should be noted that the classifier may be trained in a variety of ways (e.g., depending on the classification task), and the present disclosure is not intended to be limited to any particular classifier or training thereof.

The trained embedding generator 101 may then be used as part of the molecule classification module 105 (e.g., to output a predicted class label for a candidate molecule). The molecule classification module 105 may use the trained embedding generator 101 to classify the candidate molecule as a potentially active molecule or an inactive molecule, for example.

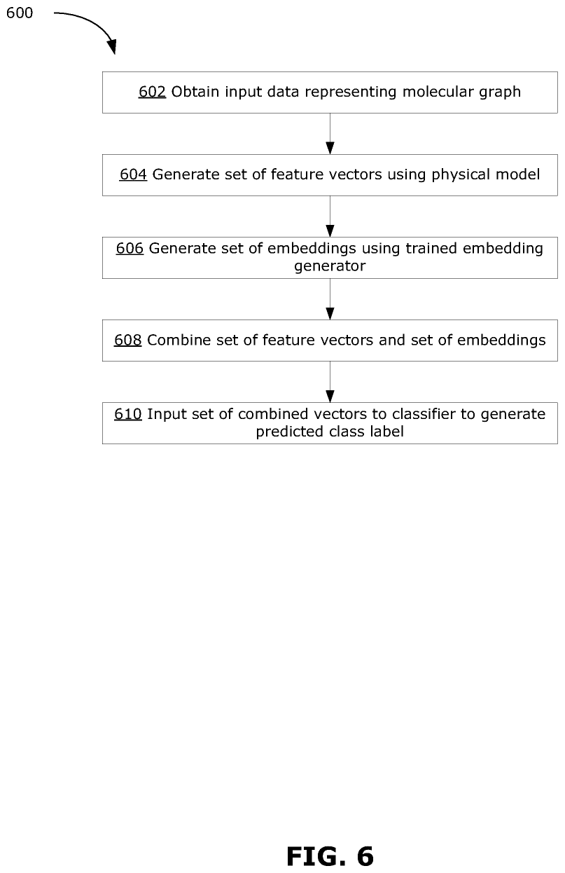

FIG. 6 is a flowchart illustrating an example method 600 for classifying a molecular graph, using the trained embedding generator 101. The method 600 may be performed by any suitable computing system. In particular, the method 600 may be performed by a computing system executing software instructions for the molecule classification module 105.

At 602, input data representing a molecular graph is obtained (e.g., an electronic file representing the molecular graph may be received as input data). The input data may, for example, be a SDF representing a candidate molecule to be classified.

At 604, the input data is provided to the physical model 202 to generate a set of task-relevant feature vectors. The set of task-relevant feature vectors represent local physical features of the molecular graph (e.g., based on a molecular docking model).

At 606, the input data is provided to the trained embedding generator 101 to generate a set of task-relevant structural embeddings. The set of task-relevant structural embeddings represent the connectivity of the vertices of the geometric graph, as well as task-relevant feature information of the vertices. The task-relevant feature information may be task-specific, for example bond order of the edges, partial electric charge at the corresponding atom, its Van-der-Waals radius, hydrogen bonding potential, among other possibilities, in the case of a molecular classification task.

Although steps 604 and 606 have been illustrated in a particular order, it should be understood that steps 604 and 606 may be performed in any order, and may be performed in parallel.

At 608, the set of task-relevant feature vectors (generated at step 604) and the set of task-relevant structural embeddings (generated at step 606) are combined to obtain a set of combined vectors. Specifically, the task-relevant feature vector corresponding to a given vertex may be concatenated with the task-relevant structural embedding corresponding to the same given vertex, to obtain a combined vector corresponding to that given vertex. In this way, a set of combined vectors is obtained corresponding to the set of vertices for the molecular graph.

At 610, the set of combined vectors is provided as input to a trained classifier 204, to generate a predicted class label for the molecular graph. In examples where the input data represents a candidate molecule (e.g., for drug discovery applications), the predicted class label may indicate whether the candidate molecule is predicted to be active or inactive.

In various examples, the present disclosure has described an approach to generate task-relevant structural embeddings of a molecular graph based on an adaption of good edit similarity, in which an encoder-decoder pair is trained to learn a latent representation of the local features of the molecular graph. In particular, a hierarchy of margins approach is used to classify local and non-local features for each vertex of the molecular graph.

The disclosed embedding generator may be used to generate input to a classifier (e.g., in a molecule classification module), or may be used separately from a classifier. In some examples, the molecular structure reconstruction loss may be used for training the classifier.

The present disclosure has described methods and systems in the context of biomedical applications, such as drug discovery applications. However, it should be understood that the present disclosure may also be suitable for application in other technological fields, including other technical applications that involve computations on geometric graphs. For example, the present disclosure may be applicable to generating embeddings for representing social graphs (e.g., for social media applications), representing urban networks (e.g., for city planning applications), or for software design applications (e.g., embeddings representing computation graphs, data-flow graphs, dependency graphs, etc.), among others. The disclosed methods and systems may be suitable, in particular, for applications in which geometric graphs exhibit local symmetry. Other such applications may be possible within the scope of the present disclosure.

A person of ordinary skill in the art may be aware that, in combination with the examples described in the embodiments disclosed in this disclosure, units and algorithm steps may be implemented by electronic hardware or a combination of computer software and electronic hardware. Whether the functions are performed by hardware or software depends on particular applications and design constraint conditions of the technical solutions. A person skilled in the art may use different methods to implement the described functions for each particular application, but it should not be considered that the implementation goes beyond the scope of this disclosure.

It may be clearly understood by a person skilled in the art that, for the purpose of convenient and brief description, for a detailed working process of the foregoing system, apparatus, and unit, refer to a corresponding process in the foregoing method embodiments, and details are not described herein again.

It should be understood that the disclosed systems and methods may be implemented in other manners. The units described as separate parts may or may not be physically separate, and parts displayed as units may or may not be physical units, may be located in one position, or may be distributed on a plurality of network units. Some or all of the units may be selected according to actual requirements to achieve the objectives of the solutions of the embodiments. In addition, functional units in the embodiments of this application may be integrated into one processing unit, or each of the units may exist alone physically, or two or more units are integrated into one unit.

When the functions are implemented in the form of a software functional unit and sold or used as an independent product, the functions may be stored in a computer-readable storage medium. Based on such an understanding, the technical solutions of this disclosure essentially, or the part contributing to the prior art, or some of the technical solutions may be implemented in a form of a software product. The software product is stored in a storage medium, and includes several instructions for instructing a computer device (which may be a personal computer, a server, or a network device) to perform all or some of the steps of the methods described in the embodiments of this application. The foregoing storage medium includes any medium that can store program code, such as a universal serial bus (USB) flash drive, a removable hard disk, a read-only memory (ROM), a random access memory (RAM), a magnetic disk, or an optical disc, among others.

The foregoing descriptions are merely specific implementations of this application, but are not intended to limit the protection scope of this disclosure. Any variation or replacement readily figured out by a person skilled in the art within the technical scope disclosed in this disclosure shall fall within the protection scope of this disclosure.

1. A method for classifying a candidate molecule, the method comprising:
obtaining input data representing a molecular graph defined by a set of vertices and a set of edges, the molecular graph being a representation of the candidate molecule;
generating, using a physical model, a set of task-relevant feature vectors from the input data, the set of task-relevant feature vectors representing local physical features of the molecular graph;
generating, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule;
combining each task-relevant feature vector in the set of task-relevant feature vectors with a respective task-relevant structural embedding in the set of task-relevant structural embeddings, to obtain a set of combined vectors; and
generating, using a trained classifier, a predicted class label for the input data from the set of combined vectors, the predicted class label representing a classification of the candidate molecule.

2. The method of claim 1, wherein the embedding generator comprises a geometrical embedder based on good edit similarity and a gated recurrent unit (GRU) module, the geometrical embedder generating a set of geometrical embeddings representing the connectivity among the set of vertices, the GRU module further generating the set of task-relevant structural embeddings from the set of geometrical embeddings and task-relevant features.

3. The method of claim 2, wherein the geometrical embedder is trained to generate the set of geometrical embeddings using a hierarchy of margins to encode local connections with respect to each vertex in the set of vertices.

4. The method of claim 2, wherein training the geometrical embedder and the GRU module comprises generating, using a decoder neural network, a reconstructed adjacency matrix of the molecular graph from the set of task-relevant structural embeddings, computing a molecular structure reconstruction loss between the reconstructed adjacency matrix and an actual adjacency matrix of the molecular graph, and backpropagating the molecular structure reconstruction loss to update weights of the GRU module and the geometrical embedder.

5. The method of claim 4, wherein the molecular structure reconstruction loss is used as a regularization term for training of the classifier.

6. The method of claim 1, wherein combining each task-relevant feature vector in the set of task-relevant feature vectors with the respective task-relevant structural embedding in the set of task-relevant structural embeddings comprises concatenating each task-relevant feature vector in the set of task-relevant feature vectors with the respective task-relevant structural embedding in the set of task-relevant structural embeddings.

7. The method of claim 1, wherein the physical model is a molecular docking model.

8. A device for classifying a candidate molecule, comprising:
a processing unit configured to execute instructions to cause the device to:
obtain input data representing a molecular graph defined by a set of vertices and a set of edges, the molecular graph being a representation of the candidate molecule;
generate, using a physical model, a set of task-relevant feature vectors from the input data, the set of task-relevant feature vectors representing local physical features of the molecular graph;
generate, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule;
combine each task-relevant feature vector in the set of task-relevant feature vectors with a respective task-relevant structural embedding in the set of task-relevant structural embeddings, to obtain a set of combined vectors; and
generate, using a trained classifier, a predicted class label for the input data from the set of combined vectors, the predicted class label representing a classification of the candidate molecule.

9. The device of claim 8, wherein the embedding generator comprises a geometric embedder based on good edit similarity and a gated recurrent unit (GRU) module, the geometric embedder generating a set of geometrical embeddings representing the connectivity among the set of vertices, the GRU module further generating the set of task-relevant structural embeddings from the set of geometrical embeddings and task-relevant features.

10. The device of claim 9, wherein the geometrical embedder is trained to generate the set of geometrical embeddings using a hierarchy of margins to encode local connections with respect to each vertex in the set of vertices.

11. The device of claim 9, wherein training the geometrical embedder and the GRU module comprises generating, using a decoder neural network, a reconstructed adjacency matrix of the molecular graph from the set of task-relevant structural embeddings, computing a molecular structure reconstruction loss between the reconstructed adjacency matrix and an actual adjacency matrix of the molecular graph, and backpropagating the molecular structure reconstruction loss to update weights of the GRU module and the geometrical embedder.

12. The device of claim 11, wherein the molecular structure reconstruction loss is used as a regularization term for training of the classifier.

13. The device of claim 8, wherein combining each task-relevant feature vector in the set of task-relevant feature vectors with the respective task-relevant structural embedding in the set of task-relevant structural embeddings comprises concatenating each task-relevant feature vector in the set of task-relevant feature vectors with the respective task-relevant structural embedding in the set of task-relevant structural embeddings.

14. The device of claim 8, wherein the physical model is a molecular docking model.

15. The device of claim 8, wherein the physical model, the trained embedding generator and the trained classifier are part of a molecule classification module executed by the processing unit.

16. A computer-readable medium having instructions encoded thereon, wherein the instructions, when executed by a processing unit of a device, cause the device to:
obtain input data representing a molecular graph defined by a set of vertices and a set of edges, the molecular graph being a representation of the candidate molecule;
generate, using a physical model, a set of task-relevant feature vectors from the input data, the set of task-relevant feature vectors representing local physical features of the molecular graph;
generate, using a trained embedding generator, a set of task-relevant structural embeddings from the input data, the set of task-relevant structural embeddings representing connectivity among the set of vertices and task-relevant features of the set of vertices, the task-relevant features being relevant for classifying the candidate molecule;
combine each task-relevant feature vector in the set of task-relevant feature vectors with a respective task-relevant structural embedding in the set of task-relevant structural embeddings, to obtain a set of combined vectors; and
generate, using a trained classifier, a predicted class label for the input data from the set of combined vectors, the predicted class label representing a classification of the candidate molecule.

17. The computer-readable medium of claim 16, wherein the embedding generator comprises a geometric embedder based on good edit similarity and a gated recurrent unit (GRU) module, the geometric embedder generating a set of geometrical embeddings representing the connectivity among the set of vertices, the GRU module further generating the set of task-relevant structural embeddings from the set of geometrical embeddings.

18. The device of claim 17, wherein the geometrical embedder is trained to generate the set of geometrical embeddings using a hierarchy of margins to encode local connections with respect to each vertex in the set of vertices.

19. The device of claim 17, wherein training the geometrical embedder and the GRU module comprises generating, using a decoder neural network, a reconstructed adjacency matrix of the molecular graph from the set of task-relevant structural embeddings, computing a molecular structure reconstruction loss between the reconstructed adjacency matrix and an actual adjacency matrix of the molecular graph, and backpropagating the molecular structure reconstruction loss to update weights of the GRU module and the geometrical embedder.

20. The device of claim 19, wherein the molecular structure reconstruction loss is used as a regularization term for training of the classifier.
